(19) United States
(12) Patent Application Publication
(10) Pub. No.: US 2010/0173008 A1
(43) Pub. Date: Jul. 8, 2010

(54) METHOD OF TREATING OR ALLEVIATING THE SYMPTOMS OF ALESION IN MAMMAL

(76) Inventor: SONG DOUG KANG, DAEJEON (KR)

Correspondence Address: J C PATENTS, 4 VENTURE, SUITE 250, IRVINE, CA, 92618, US

(21) Appl. No.: 12/727,991

(22) Filed: Mar. 19, 2010

Related U.S. Application Data
(62) Division of application No. 10/580,741, filed on May 24, 2006.

(30) Foreign Application Priority Data
Nov. 24, 2003 (KR) 10-2003-0083814

(51) Int. Cl. A61K 33/00; A61P 19/02; A61P 11/06; A61P 25/16; A61P 27/02; A61P 9/00; A61P 3/10

(52) U.S. Cl. 424/600

(57) ABSTRACT

Provided is a new use of Brown's gas, more particularly, to the use of Brown's gas for the treatment of diseases in mammals and a Brown's gas supply apparatus therefor. It is based on the assumption that the Brown's gas is a medium capable of directly supplying moisture (special form of water according to the third theory of Brown's gas) to many regions of the body via skin, etc. Various effects of the Brown's gas on the body have been demonstrated. In particular, provided is a new use of the Brown's gas for treatment or alleviation of the symptoms of a lesion in a lesional tissue, cell, or organ of a mammal. Provided is also a simple and inexpensive Brown's gas supply apparatus for supplying pure Brown's gas suitable to be applied to the human body.

### TECHNICAL FIELD

The present invention relates to a new use of Brown's gas, and more particularly, to the use of Brown's gas for the treatment of diseases in mammals and a Brown's gas supply apparatus therefor.

### BACKGROUND ART

Brown's gas is a mixed gas of hydrogen and oxygen (2:1, v/v) created by electrolysis of water. It is thought that Brown's gas also contains considerable water vapor. Generally, electrolysis of water produces a hydrogen gas at cathode and an oxygen gas at anode. These gases are captured at the same time without being separated, and the captured mixed gas is generally known as “Brown's gas”. Brown's gas has several characteristic properties, unlike general gases. The most noticeable property of Brown's gas is implosion upon ignition. For this reason, Brown's gas is known to have ultra-high temperature to an extent that can sublimate tungsten with the highest melting point of all metals.

According to the classic theory (the first theory), Brown's gas is a simple mixed gas of hydrogen and oxygen generated by electrolysis of water, which cannot explain the special characteristics of Brown's gas. Accordingly, new theories about the entity of Brown's gas have been recently introduced. Experimental results acquired to date show that a mixture of oxygen and hydrogen generated by a Brown's gas generator has remarkably larger reactivity than an equal amount of another mixture of oxygen and hydrogen. The second theory about Brown's gas is that Brown's gas is a mixture of diatomic hydrogen/oxygen and monatomic hydrogen/oxygen [Brown's Gas Book One; www.watertorch.com]. The recently published third theory is that electrolysis of water produces third gas bubbles between a cathode and an anode, in addition to a molecular hydrogen gas at the cathode and a molecular oxygen gas at the anode. According to the third theory, it is thought that Brown's gas is a special water gas in which the hydrogen gas, the oxygen gas, and the third gas bubbles are mixed, i.e., Brown's gas is not steam but “electrically expanded water” [Brown's Gas Book Two; www.watertorch.com].

The characteristics of Brown's gas disclosed to date are as follows. Brown's gas contains high energy, and in particular, generates cold flame with tremendous energy potential upon ignition. Brown's gas is a special and highly efficient medium that transmits electrical energy to the atomic structure of a material and exhibits special effects that are often unobtainable by any other means. Brown's gas is essentially easy to handle, is odorless, and is harmless to the human body even when inhaled. Further, Brown's gas is lighter than air, diffuses rapidly in air, and has a high initial flammability limit, which makes it safer than other combustible gases. Due to these characteristics, Brown's gas has received interest as a next generation fuel, in particular, as a clean fuel that produces no pollutants, unlike a waste fuel producing pollutants, and thus, research into utility of Brown's gas as a fuel has been actively conducted.

The present inventor has approached Brown's gas from a different standpoint from studies conducted to date, and thus, the present invention provides a new use of Brown's gas.

Water is a material occupying the largest proportion of the human body among components constituting the human body and occupies about 50-60% by weight of the human body. The proportion of water in the human body significantly varies according to age and sex. Newborn male babies have about 80% water with females over the age of 60 having about 45% water. Generally, the proportion of water in the weight of males is larger than that of females. The proportion of water in the human body decreases with age regardless of sex. However, it is noted that even in a healthy person, the amount of water in the body decreases as aging continues. This shows that water is associated with human aging.

Water makes up body fluids and is an important component in various tissues and organs as well. The action of water in the human body currently known is as follows.

(A) Transfer System

Blood, which is 83% water, functions as a transfer system in the human body and transports nutrients, hormones, enzymes, oxygen, and other vital material to cells. Blood also carries waste matters to internal organs such as the kidneys or lungs to discharge the waste matters from the human body. Foods are digested to the simplest nutrients in the stomach and intestines, absorbed in blood and lymph, and then transported to cells. Inhaled oxygen by breathing is absorbed in blood by gas exchange in the lungs and transported to cells. Water constitutes most of blood and lymph which are transport systems.

(B) Main Component of Various Secretions

Various secretions are produced in the human body to facilitate the metabolic action of the human body. These secretions are mainly made up of water.

(C) Lubricant

Abundant water is contained in fluids surrounding various internal organs of the human body or mucous linings of the organs. These fluids or mucous linings reduce friction in the human body to promote the movements of internal organs. Further, joints are filled with synovial fluid containing a large volume of water. Therefore, when bones of the joints move, the synovial fluid reduces friction between the bones of the joints.

(D) Digestion

Water is a main component of viscous liquid of the digestive tract or saliva. The viscous liquid of the digestive tract and saliva assist digestion of foods and the digested foods are transported to tissues through the digestive tract.

(E) Body Temperature Regulation

Human beings are warm-blooded animals and thus must maintain an almost constant body temperature. Water plays a very important role in keeping the human body at a constant temperature. That is, it is known that water is a good thermal conductor and the specific heat of water is the highest of all solids and liquids except ammonia (liquid). The phrase “a substance has a high specific heat” indicates that the substance undergoes a smaller increase in temperature than another substance when the same amount of heat is added. Due to the high specific heat of water, the temperature of the human body is not easily increased even when much heat is given off from the human body by exercise, or the like. Further, when heat is given off from the human body, the human body promotes sweat secretion to prevent the increase of its body temperature. This is possible because the evaporation of sweat cools the human body by removing latent heat. Since the evaporation latent heat of water is the highest of all substances, a body temperature regulation by cooling of the human body can be efficiently achieved.

(F) Chemical Reaction in the Human Body

Various types of chemical reactions occur in tissues and cells of the human body. Water assists the occurrence of these chemical reactions or directly participates in these chemical reactions. For example, it is known that water plays a very important role in digestion of proteins and carbohydrates and assists the synthesis of hormones and enzymes that regulate chemical reactions occurring in the human body. In this regard, the absence of water cannot trigger most chemical reactions in the human body, thereby losing life-sustaining functions.

(G) Removal of Waste Matters

Waste matters are produced in the human body by various routes. Water takes a major role in discharge of waste matters thus produced out of the body via the kidneys and the large intestine. Waste matters are also discharged out from the body through sweat or air to be exhaled during breathing.

(H) Protection

Water is elastic, and thus, serves to cushion impact applied to the body to decrease damage to the internal organs or tissues of the body. It is known that central nervous tissues are immersed in and protected by the cerebral spinal fluid.

(I) Physiological Actions

Currently recognized physiological actions of water in the human body are as follows.

(a) Water assists the body in detoxification, resulting in a reduced burden on the liver and kidneys.

(b) Water eliminates the viscosity of blood and prevents the stroke.

(c) Water intake is effective for treatment and prevention of constipation.

(d) Water increases the immunity of the nasal mucous membrane and thus prevents cold. “Sufficient water intake” is typically prescribed to relieve medical conditions such as high fever, cold, and diarrhea. Water has been regarded as a good anti-cold strategy since it makes the bronchial mucous membrane wet and relieves high fever and inflammation. Also, in modern medical science, many doctors encourage cold patients to drink sufficient water frequently.

(e) Sufficient water intake before drinking liquor can reduce the burden on the liver and the degree of headache after overdrinking.

(f) Water intake is effective for prevention or relief of nephritis. It is well known that sufficient water intake is a strategy effective in relieving the symptoms of urinary tract infection such as nephritis, urethritis, and cystitis, and preventing the recurrence of the urinary tract infection. A recent report shows that a person drinking more than 2.5 quarters of water per day (1 quarter is about 0.946 liters) has less likelihood of developing cystitis, relative to a person drinking half of the more than 2.5 quarters of water per day.

(g) Water intake is effective in prevention of colon cancer. It is reported that a person drinking more than five glasses of water per day has a 45% less likelihood of developing colon cancer, relative to a person drinking less than two glasses of water per day.

(h) Water intake is effective in preventing the formation of calculi. Sufficient water intake can prevent new calculus formation in a person with renal calculi.

(i) Water dilutes toxic substances. Generally, it is known that damage caused by a toxic substance, including a carcinogenic substance, is mainly proportional to the concentration of the toxic substance, rather than the total amount. In this regard, even when the body is exposed to the same amount of a toxic substance, increased water intake can minimize damage caused by the toxic substance.

(j) Water reduces the irritation of the gastric or duodenal ulcer on an empty stomach.

(k) When someone suffers from asthmatic spasm, water intake keeps his breathing calm and relieves congestion.

(l) An allergic disease can be easily treated by sufficient water intake that induces excretion of the allergen.

(m) Sufficient water intake after the active motion of the body helps fast removal of fatigue.

### DISCLOSURE OF THE INVENTION

### Technical Problem

The present invention provides an effect of Brown's gas on the human body, i.e., a new use of Brown's gas, and is based on the following three facts.

First, according to the facts disclosed to date, Brown's gas is harmless to the human body.

Second, according to the recently published third theory, Brown's gas is a special “water gas”, i.e., “electrically expanded water”, which is an activated (high energy) form of water.

Third, water occupies about 50-60% by weight of the human body and is a material occupying the largest proportion of the human body among components constituting the human body. Water plays many important roles in the body, including many physiological functions.

Based on these facts, the present inventor assumed that Brown's gas is a medium capable of directly supplying moisture (special form of water according to the third theory of Brown's gas) to many regions of the body via skin, etc, and deduced that the Brown's gas can be effectively used for treatment of many diseases in the body. Thus, the present inventor completed the present invention.

In the present invention, many effects of Brown's gas on the body will be provided. It is thought that most of these effects are directly or indirectly associated with the action and efficacy of water in the body.

### Technical Solution

In detail, the present invention provides a new use of Brown's gas for the treatment or alleviation of the symptoms on a lesional tissue, cell, or organ of a mammal.

The Brown's gas may be locally sprayed on the lesional tissue, cell, or organ, its related region, or its adjacent skin.

At this time, the lesion may be selected from the group consisting of myalgia, arthritis, rheumatoid arthritis, disc, infectious inflammation, non-infectious inflammation, allergy, rhinitis, bronchitis, asthma, myasthenia, neuralgia, headache, bacterial ocular disease such as sty, viral ocular disease such as epidemic keratoconjunctivitis, ocular disease such as glaucoma and cataract, Parkinson's disease, gout, panic disorder, diabetic peripheral neuropathy, blood circulation dysfunction, cardiovascular disease such as stricture of the heart, and various pain such as toothache and menstrual pain.

The present invention also provides a use of Brown's gas for the treatment of a disease associated with moisture deficiency or dehydration in a lesion tissue, cell, or organ of a mammal. At this time, Brown's gas may be locally sprayed on the lesion tissue, cell, or organ, its related region, or its adjacent skin.

The present invention also provides a use of Brown's gas for moisture supply to a mammalian skin. At this time, Brown's gas may be locally sprayed on the skin.

The present invention also provides a Brown's gas supply apparatus for the above-described new use of Brown's gas, i.e., an apparatus for supplying purified Brown's gas suitable for direct spraying on mammals, including human beings.

A commercially available Brown's gas generator uses sodium hydroxide (NaOH) or potassium hydroxide (KOH) as a catalyst to facilitate the generation of oxygen and hydrogen by electrolysis of water. Therefore, Brown's gas generated by a conventional Brown's gas generator contains impurities such as NaOH and KOH, in addition to pure Brown's gas, which is not suitable for therapeutic use in mammals.

Therefore, the present invention provides a relatively simple and inexpensive apparatus for supplying purified Brown's gas suitable for use in the body.

To this end, in the present invention, there is provided a Brown's gas supply apparatus, including: a Brown's gas generator for generating Brown's gas by electrolysis of water; at least one airtight gas-trapping tank for allowing the Brown's gas to pass through water and trapping pure Brown's gas; and a spraying nozzle connected to the gas-trapping tank to spray the trapped Brown's gas on a lesional region of a mammal.

Preferably, the Brown's gas supply apparatus comprises serially connected two gas-trapping tanks, wherein the generated Brown's gas passes through water in a gas-trapping tank and is primarily trapped, and wherein the primarily trapped Brown's gas passes through water in the other gas-trapping tank and is secondarily trapped.

In addition, an elastic cap is detachably installed to an end of the spraying nozzle to prevent inflow of ambient air.

In a preferred embodiment of the present invention, the gas-trapping tank has a hermetically sealed tube structure in which water is contained in a tube, wherein a porous partition is horizontally installed in a lower part of the tube in such a way to be spaced predetermined distance apart from the bottom of the tube so that the Brown's gas and the water can pass through the porous partition, wherein an upper surface of the gas-trapping tank is formed with a gas injection pipe extended downward to below the porous partition to supply the Brown's gas to a lower part of the tube; a gas discharge port for discharging the trapped Brown's gas; and a water injection port for water supply, and wherein the Brown's gas, injected into the water below the porous partition via the gas injection pipe, passes through the porous partition and the water and is trapped in an upper part of the tube.

In another embodiment of the present invention, the gas-trapping tank has a hermetically sealed tube structure in which water is contained in a tube, wherein an upper surface of the gas-trapping tank is formed with a gas injection pipe extended downward to supply the Brown's gas to a lower part of the tube, a gas discharge port for discharging the trapped Brown's gas, and a water injection port for water supply, and wherein the Brown's gas, injected into the water in the lower part of the tube via the gas injection pipe, passes through the water and is trapped in an upper part of the tube.

In still another embodiment of the present invention, the gas-trapping tank has a hermetically sealed dual tube structure and comprises an inner tube, an outer tube separated from a sidewall and bottom of the inner tube by a predetermined distance, and a common upper plate covering the inner tube and the outer tube, the inner tube and the outer tube containing water, wherein the bottom of the inner tube is formed with a plurality of through-holes through which the Brown's gas and the water can pass, wherein the upper plate is formed with a gas injection pipe extended downward to supply the Brown's gas generated in the Brown's gas generator to water in the outer tube; a gas discharge port for discharging the trapped Brown's gas, and a water injection port for water supply, and wherein the Brown's gas, injected into the water in the outer tube via the gas injection pipe, passes through the water in the inner tube via the through-holes, and is trapped in an upper part of the inner tube.

### BRIEF DESCRIPTION OF THE DRAWINGS

FIG. 1 is a perspective view of a Brown's gas supply apparatus according to an embodiment of the present invention.

FIG. 2 is a sectional view of an example of an airtight gas-trapping tank of FIG. 1.

FIG. 3 is a sectional view of another example of an airtight gas-trapping tank of FIG. 1.

FIG. 4 is a partial sectional view of a Brown's gas supply apparatus according to the embodiment shown in FIG. 3.

FIGS. 5A and 5B are X-ray images before and after therapy in a patient with degenerative arthritis in Example 1

### BEST MODE FOR CARRYING OUT THE INVENTION

Hereinafter, embodiments of the present invention will be described in detail with reference to the attached drawings. The present invention is not restricted to the following embodiments, and many variations are possible within the spirit and scope of the present invention. The embodiments of the present invention are provided in order to more completely explain the present invention to anyone skilled in the art.

Efficacies of Brown's gas currently found by the present inventor are as follows.

1. Elimination or Alleviation of Myalgia

When Brown's gas was sprayed on the region of the body where myalgia, frozen shoulder, muscular stiffness such as neck stiffness, or bruises is located, alleviation or elimination of pain by rapid relaxation of a muscle was observed. Further, a bruise at a bruised region disappeared and normal skin color was regained.

These phenomena can be explained as follows: sprayed Brown's gas is absorbed in skin and hydrates the muscle where pain occurs to recover the tissues of the muscle, resulting in elimination of the pain by relaxation of the muscle.

2. Alleviation of Symptoms and Pain of Rheumatoid Arthritis, Degenerative Arthritis, etc

When Brown's gas was sprayed onto the lesional regions of patients who suffered from rheumatoid arthritis, knee arthralgia, wrist arthralgia, and knuckle arthralgia, the patients felt less pain and that the joints were relaxed. Acute pain was alleviated by spraying occurred only once. Some patients underwent pain alleviation so that they had a normal life. These therapeutic effects continued for a considerably extended period of time. Furthermore, in patients who suffered from an aftereffect of knee joint surgery (cruciate ligament implant surgery, cartilage implant surgery), recovery of joint function, together with pain alleviation, was observed. Improvement in the symptoms of Achilles myositis was also observed.

Like for myalgia, these phenomena can be explained as follows: Brown's gas is absorbed through skin and supplies moisture to joint synovial fluid to lubricate joints. There is another interpretation based on the second theory of Brown's gas that monatomic hydrogen contained in Brown's gas serves as active hydrogen in the body. That is, rheumatoid arthritis is known to be an autoimmune disease that spontaneously creates an inflammatory substance that attacks joint synovium, causing joint inflammation. High level of the inflammatory substance intensifies pain, whereas low level of the inflammatory substance alleviates pain. Even though the creation of the inflammatory substance is attributed to a genetic factor and an environmental factor, the gene of interest has not been identified. However, it is known that active oxygen plays an important role in the creation of the inflammatory substance. A serum test shows a remarkably reduced level of an antioxidative ingredient in a patient with rheumatoid arthritis, relative to a normal person. In this respect, many attempts have been made to use an antioxidant as an anti-inflammatory agent. Thus, it is thought that active hydrogen, known to serve as an antagonist to active oxygen in the body, alleviates rheumatoid arthritis. Further, the spraying of Brown's gas improved the node and inflammation by gout, which can also be explained similarly to that described above.

3. Alleviation of the Symptoms of Intervertebral Disc

Like for arthritis, patients with intervertebral disc experienced alleviation of pain and symptoms after spraying Brown's gas on a lesional region. The alleviation of pain and symptoms is attributed to hydration of the lesional tissue of intervertebral disc by Brown's gas absorbed through skin.

4. Elimination or Alleviation of Headache

When Brown's gas was sprayed on the necks of patients with severe headache, including chronic headache, migraine, and acute headache by impact applied to the cervical vertebrae in car accident, the patients had a clear head and rapid alleviation or elimination of headaches. Further, the therapeutic effect continued over a prolonged period of time (six months, at present) after therapy with Brown's gas.

These phenomena can be explained as follows: sprayed Brown's gas is absorbed in neck skin and hydrates neck muscles. Therefore, muscle tissues are relaxed and thus contracted blood vessels are restored to normal. As a result, blood flow increases and sufficient oxygen supply to the brain is achieved, resulting in disappearance of headache.

5. Elimination of Inflammation in Wounded Region and Body Region Affected by Bacterial Inflammation, or Rapid Recovery of the Affected Regions

When Brown's gas was directly sprayed on a wounded region of the body, it was observed that the wound was rapidly cured. Furthermore, when Brown's gas was sprayed on the body region affected by bacterial inflammation such as laryngitis, otitis media, and sinus infections (ozena), alleviation or rapid elimination of inflammation was observed. This might be possible because Brown's gas destructs or prevents the growth of anaerobic microorganism in the wounded region and various microorganisms in the body region affected by inflammation, and at the same time, helps cell restoration in the wounded region and the body region affected by inflammation. The destruction and prevention of the growth of microorganisms can be sufficiently explained assuming that Brown's gas contains large amounts of reactive oxygen, in particular monatomic and diatomic oxygen based on the second theory of Brown's gas.

6. Prevention of Inflammatory and Allergic Cutaneous Reaction

When Brown's gas was sprayed on the skin where allergic reaction occurred, complete disappearance of allergy symptoms was observed. Furthermore, when Brown's gas was sprayed on the skin where non-infectious inflammatory reaction occurred, remarkable improvement of inflammation was observed by spraying occurred only once. It was also observed that the spraying of Brown's gas is effective for the treatment of eye's movement relating nerve disorder due to inflammation in nerve cells at the back part of the head. These therapeutic effects are attributed to cell restoration by hydration of target skin cells by Brown's gas.

It is expected that the effect of Brown's gas on skin can be extended to skin tissue restoration, moisturizing, anti-aging, etc. Therefore, Brown's gas can also be used as a skin-moisturizing agent for cosmetic purpose.

7. Pain Removal or Alleviation

When Brown's gas was applied to patients with sciatica, pain in right pelvis, pain by the bone fracture of ankle joint, pain by the bone fracture of wrist joint, pain by herpes shingles, toothache, menstrual pain, etc., considerable pain alleviation was observed.

8. Ocular Disease

Patients with ocular diseases such as simple ocular congestion and afterimage, sties, epidemic keratoconjunctivitis, glaucoma, and cataract experienced considerable disease improvement effects by spraying of Brown's gas.

9. Parkinson's Disease

When Brown's gas was sprayed on patients with Parkinson's disease, considerable improvement in paralysis of limbs, speech disorder, dribbling by excessive salivation, head shaking, trembling of hands, etc. was observed.

Furthermore, symptoms removal, improvement, or relief in insomnia by nervousness, myasthenia by muscle weakness, chilly hands and feet by blood circulation dysfunction, asthma, gastritis and gastric ulcer, panic disorder, diabetic peripheral neuropathy, trouble in the left chest by stricture of the heart, breathing discomfort, etc. was observed.

In addition to cure or improvement of symptoms during or after therapy with Brown's gas, patients experienced the following common effects.
Lightness and clearness of the head.
Lightness of the body and good condition.
Disappearance of the dimness and redness in the eyes.
Warm hands and feet and perspiration.
Good sleep.

These effects are attributed to promoted blood circulation by spraying of Brown's gas, resulting in smooth blood supply to various regions in the body.

It is thought that the aforementioned many effects of Brown's gas on the body are closely related to the action and physiological efficacy of water in the body. Therefore, the present inventor thinks that a basic mechanism that can explain the effect of Brown' gas on the body is absorption of Brown's gas through skin, moisture supply and hydration for target tissues, cells, or organs.

However, the effect of Brown's gas on the aforementioned various diseases cannot be explained only by simple hydration. Therefore, the present inventor thinks that there will be a more complicated therapeutic mechanism model of Brown's gas. Thus, the present inventor presumes that basic therapeutic mechanism of Brown's gas is strong immunity and activation of the body by smooth blood circulation which are induced by transfer of high energy to a lesional region. In summary, from microscopic points of view, it is thought that high energy transformed into a water gas form called Brown's gas hydrates cell tissues, which activates cells, resulting in cure of lesions.

A Brown's gas generator that can be used herein may be a commonly known Brown's gas generator with a capacity appropriate to be used in the body. A commercially available common Brown's gas generator includes a “mixed gas generator” for generating a highly pure mixed gas of oxygen, hydrogen, and water vapor by electrolysis of water and a “spraying nozzle” connected to the mixed gas generator to spray the mixed gas. The spraying nozzle of the common Brown's gas generator can be used according to the present invention to directly spray Brown's gas to a lesional region of the body or its adjacent region. However, since the common Brown's gas generator uses a catalyst (NaOH, KOH etc.), considerable amounts of impurities such as NaOH or KOH, together with pure Brown's gas, are contained in Brown's gas to be supplied. In this respect, to use Brown's gas according to the present invention, it is preferable to use a Brown's gas supply apparatus equipped with a purification unit capable of removing these impurities. It is particularly preferable to use a Brown's gas supply apparatus according to the present invention as will be described later.

The present invention includes all embodiments that use Brown's gas for the purpose of the present invention regardless of the type or shape of a Brown's gas supply apparatus. Therefore, Brown's gas of the present invention cannot be construed to be limited to that supplied by the following apparatus. Also, Brown's gas supplied by various types of Brown's gas supply apparatuses that will be developed for use in the body in future must be construed to fall within the scope of the present invention provided that it is used for the purpose of the present invention.

Hereinafter, exemplary embodiments of Brown's gas supply apparatuses according to the present invention will be described with reference to the accompanying drawings. The detailed description of the same constitutional elements as in a common Brown's gas generator will be omitted without departing from the technical points of the present invention. Further, even though constitutional elements in the accompanying drawings are represented by reference numerals to assist the understanding of the present invention, the present invention is not limited to or by them.

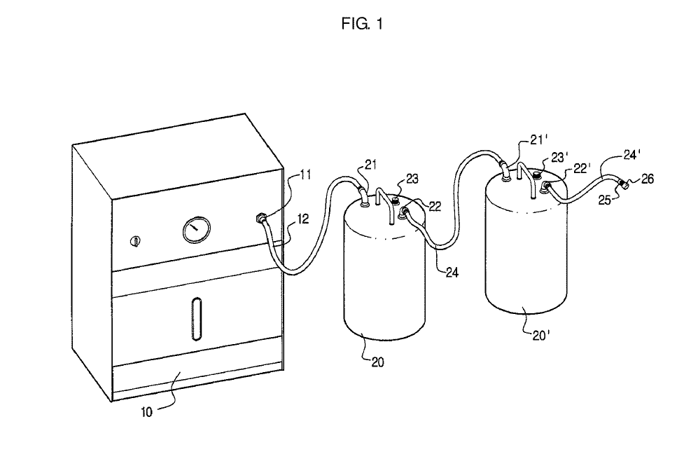

FIG. 1 is a perspective view of a Brown's gas supply apparatus according to an exemplary embodiment of the present invention. Referring to FIG. 1, the Brown's gas supply apparatus according to the embodiment of the present invention includes a Brown's gas generator 10 for generating Brown's gas by electrolysis of water; at least one airtight gas-trapping tank 20 for allowing the Brown's gas to pass through water and trapping pure Brown's gas; and a spraying nozzle 25 connected to the gas-trapping tank to spray the trapped Brown's gas on a target region.

A commonly known Brown's gas generator may be used as the Brown's gas generator 10. The capacity of the Brown's gas generator 10 is determined according to the number of patients to be treated at the same time. When patients are individually treated, the capacity of the Brown's gas generator 10 is sufficient to be 1,200/hr. In illustrative examples of the present invention as will be described later, ER1200 model (Water Torch, U.S.A.) was used.

The spraying nozzle 25 may be a commonly known nozzle such as a convergent nozzle and a divergent nozzle. It is preferable that the spraying nozzle 25 has a continuous spraying function. When needed, a nozzle with various pulsing functions may be used. An elastic cap 26 made of silicon rubber to prevent the inflow of ambient air may be detachably installed to an end of the nozzle 25.

As to the gas-trapping tank, it is preferable that two gas-trapping tanks are serially connected, as shown in FIG. 1. Brown's gas trapped in a first gas-trapping tank 20 is supplied to a second gas-trapping tank 20′ and passes through water in the second gas-trapping tank 20′ to thereby trap pure Brown's gas.

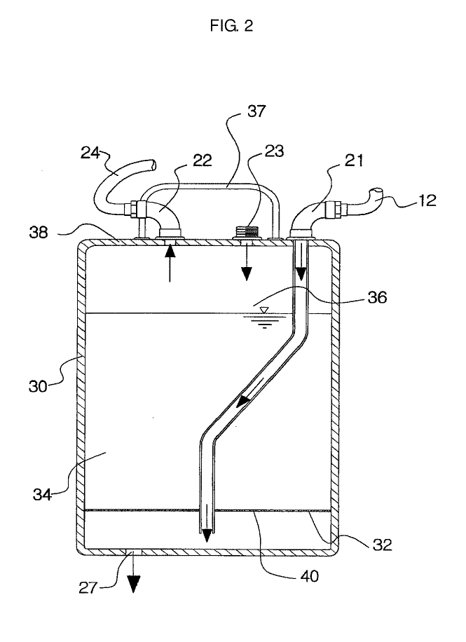

FIG. 2 is a sectional view of an example of the gas-trapping tank 20 of FIG. 1. The gas-trapping tank according to the embodiment of the present invention has a hermetically sealed tube structure in which water is contained in a tube 30. Generally, water 34 occupies 40-90% of the tube 30, preferably 50-60%. The water 34 may be clear room-temperature water or tepid water. When two gas-trapping tanks are used, it is preferable that distilled water is contained in a second gas-trapping tank. A porous partition 40 is horizontally installed in a lower part of the tube 30 in such a way to be spaced predetermined distance apart from the bottom of the tube 30 so that Brown's gas and the water 34 can pass through the porous partition 40. The porous partition 40 is responsible for dispersion of Brown's gas. An upper surface (upper plate) 38 of the tube 30 is formed with a gas injection pipe 21 extended downward to below the porous partition 40 to supply Brown's gas to a lower part of the tube 30; a gas discharge port 22 for discharging trapped Brown's gas; and a water injection port 23 for water supply. Further, a lower surface (lower plate) of the tube 30 may be formed with a water discharge port 27 for easy water change. A handle 37 may be attached to the upper surface 38 of the tube 30 to facilitate the movement of the gas-trapping tank. Even though there is no particular limitation on the material for the tube 30, the tube 30 may be made of anticorrosive stainless steel.

Referring to FIGS. 1 and 2, Brown's gas generated by the Brown's gas generator 10 is injected into the water 34 below the porous partition 40 in the first gas-trapping tank 20 via the gas injection pipe 21. The injected Brown's gas is trapped in an upper part 36 of the tube 30 after passing through the porous partition 40 and the water 34. Primarily purified Brown's gas thus trapped is injected into water below a porous partition in the second gas-trapping tank 20′ via a gas injection pipe 21′ and the same procedure as in the first gas-trapping tank 20 is performed to trap pure Brown's gas for therapeutic purpose.

The porous partition 40 in the embodiment shown in FIG. 2 may be omitted. Therefore, the absence of the porous partition 40 constitutes another embodiment (not shown) of the present invention. According to the embodiment of the present invention including no porous partitions, the diffusion effect of Brown's gas may be reduced relative to the embodiment of FIG. 2 but there is an advantage that the construction of a gas-trapping tank can be more simplified. The embodiment of the present invention including no porous partitions is the same as in the embodiment of FIG. 2 except the absence of the porous partition 40. Brown's gas is injected into water in a lower part of a tube via a gas injection pipe 21 extended downward, passes through the water, and then is trapped in an upper part of the tube. At this time, it is preferable that the gas injection pipe 21 is extended downward to an extent that nearly contacts with the bottom of the tube.

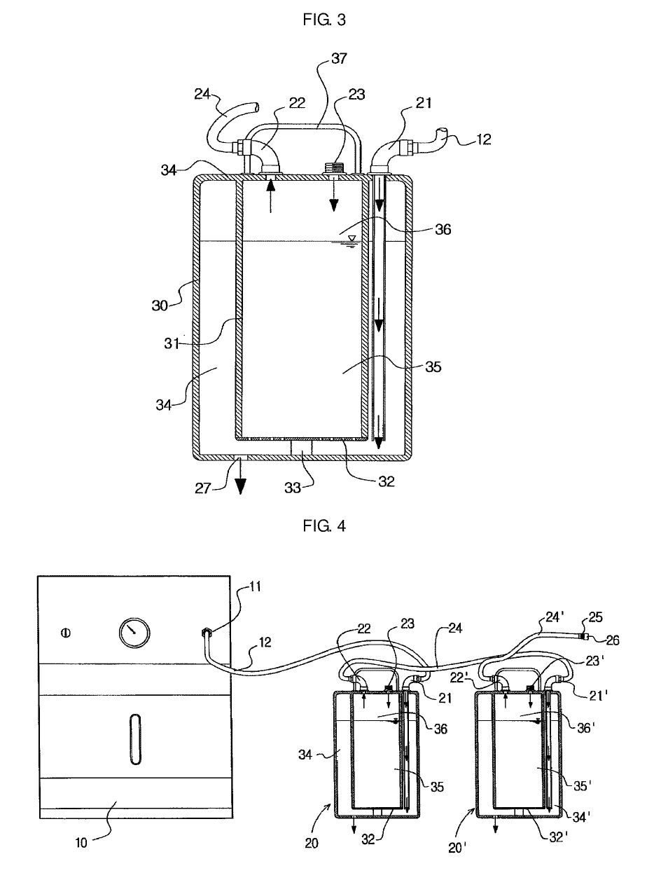

FIG. 3 is a sectional view of another example of the gas-trapping tank 20 of FIG. 1. The gas-trapping tank according to the embodiment of the present invention has a dual tube structure including an inner tube 31; an outer tube 30 separated from a sidewall and bottom of the inner tube 31 by a predetermined distance; and a common upper plate 38 covering the inner tube 31 and the outer tube 30. The inner tube 31 and the outer tube 30 contain water. The bottom of the inner tube 31 is formed with a plurality of through-holes 32 through which Brown's gas and water can pass. The upper plate 38 is formed with a gas injection pipe 21 extended downward to supply Brown's gas supplied from outside to water in the outer tube 30; a gas discharge port 22 for discharging trapped Brown's gas; and a water injection port 23 for water supply. At this time, to allow almost 100% of injected Brown's gas to pass through water, it is preferable that the gas injection pipe 21 is extended downward to an extent that nearly contacts with the bottom of the outer tube 30. The inner tube 31 can be more firmly fixed to the outer tube 30 by installing a bolt 33 between the bottom of the inner tube 31 and the bottom of the outer tube 30.

FIG. 4 is a partial sectional view of a Brown's gas supply apparatus according to the embodiment shown in FIG. 3. Specifically, FIG. 4 illustrates that Brown's gas generated by a Brown's gas generator 10 is injected in gas-trapping tanks 20 and 20′ and then pure Brown's gas is discharged from gas-trapping tanks 20 and 20′. Brown's gas generated by the Brown's gas generator 10 is supplied to a gas injection pipe 21 of a first gas-trapping tank 20 via a discharge port 11 and a discharge pipe 12. The supplied Brown's gas is injected into water 34 in an outer tube 30 of the first gas-trapping tank 20. Then, the Brown's gas passes through water 35 in an inner tube via through-holes 32 of the bottom of the inner tube and then is trapped in an upper part 36 of the inner tube. The trapped Brown's gas is discharged through a gas discharge port 22. The Brown's gas discharged after the primary purification is supplied into a gas injection pipe 21′ of a second gas-trapping tank 20′ via a discharge pipe 24 and then injected into water 34′ in an outer tube. The injected Brown's gas passes through water 35′ in an inner tube via through-holes 32′ of the bottom of the inner tube and then is trapped in an upper part 36′ of the inner tube. The Brown's gas trapped after the secondary purification is directly sprayed onto skin or lesional regions of mammals, including human beings, via a spraying nozzle 25, for therapeutic purpose.

The aforementioned embodiments have been described in terms of serially connected two gas-trapping tanks. However, serially connected three or more gas-trapping tanks may also be used. As more gas-trapping tanks are used, purer Brown's gas can be obtained. However, to obtain Brown's gas suitable for treatment considering cost and complexity, it is preferable to use two gas-trapping tanks. Further, to obtain pure Brown's gas, it is preferable to periodically change water in a gas-trapping tank.

Hereinafter, the present invention related to a new use of Brown's gas will be described more specifically by Examples. However, the following Examples are provided only for illustrations and thus the present invention is not limited to or by them.

Brown's Gas Generator

In the following Examples, a Brown's gas supply apparatus as shown in FIG. 1 was used. At this time, ER1200 model (Water Torch, U.S.A.) was used as a Brown's gas generator (10 of FIG. 1). Specification of the Brown's gas generator used was as follows:

Capacity: 1200/h

Inner pressure: 0-0.7 kg/cm²

Frequency: 45-65 Hz

Power: 200-250 VAC

Dimensions: 34.9 cm×52.7 cm×76.2 cm

Weight: 77.1 kg

### Symptoms and Results in Examples

In the following all Examples, symptoms of diseases and therapeutic results thereof were provided based on the subjective symptoms of patients.

### Example 1

Subject: female with the age of 72

Symptoms: degenerative arthritis of two knees; severe pain and stiffness in standing and walking, and numbness.

Treatment and Effect:

(1) Brown's gas was sprayed on the right knee joint for 30 minutes and on the left knee joint for 30 minutes with a 20-minute pause between the two spraying. Immediately after the spraying, the joints were relaxed and the pain completely disappeared. The patient could walk at a brisk pace on a flat ground with no assistance and did not feel severe pain when she went up and down the stairs.

(2) At the next day after the treatment, the patient felt pain in the knee joints in walking even though the level of the pain was remarkably slight, showing that the therapeutic effect was maintained during the post-treatment period.

(3) At three days after the treatment, a secondary treatment was performed in the same manner as in the section (1).

(4) At two weeks after the secondary treatment, the therapeutic effects were maintained to an extent that the knee joints were relaxed and almost no pain was felt.

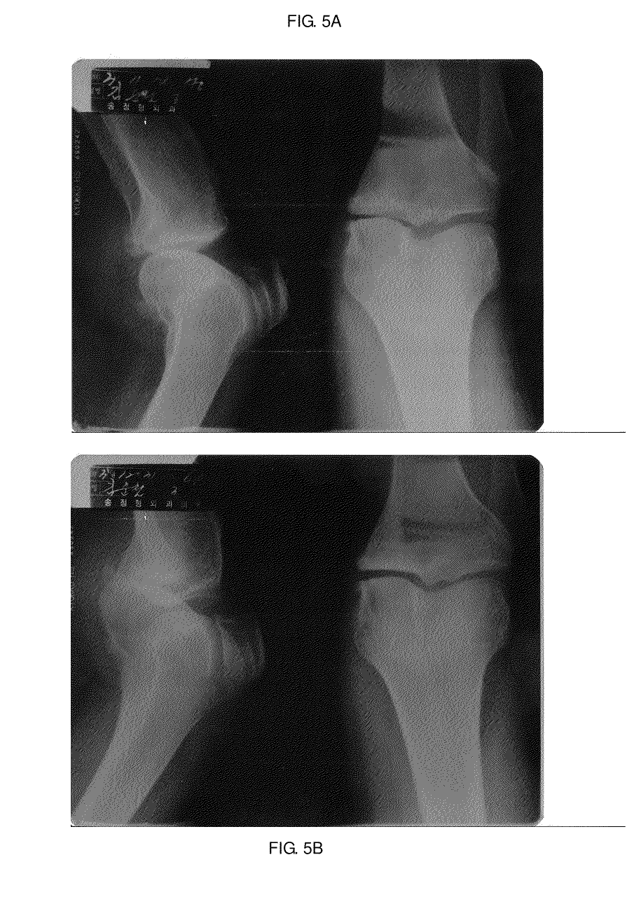

The therapeutic effects were identified by X-ray images before and after the treatment with Brown's gas. FIG. 5A is an X-ray image before the primary treatment of the section (1) (Nov. 21, 2003) and FIG. 5B is an X-ray image at two weeks after the secondary treatment of the section (3) (Dec. 31, 2003).

### Example 2

Subject: female with the age of 54

Symptoms: disc symptoms: numbness in the left hand (usual), continued loss of sensation (often) and the fifth and sixth cervical disc fusion (according to the orthopedic diagnosis); severe pain in the left shoulder and arm and stiffness in movement of the arm.

Treatment and Effect:

1. Primary Treatment and Effect

(1) First, Brown's gas was sprayed on the fifth and sixth cervical vertebrae for about 15 minutes.

(2) After 5 minutes of the spraying, knuckles were relaxed and the numbness of the hands was gradually alleviated. Pain of the left shoulder disappeared so that the patient could raise her left hand and draw a circle.

(3) Additionally, the spraying of Brown's gas was performed for 45 minutes three times (15 minutes for each). The numbness of the hands completely disappeared within one hour after the treatment. Sensation of the tips of the left knuckles was recovered.

(4) At the next day after the treatment, the improved symptoms were maintained.

2. Secondary Treatment and Effect

(5) At one week after the primary treatment, the patient complained of similar pain before the primary treatment. Brown's gas was sprayed on the shoulder and the fifth and sixth cervical vertebrae for about 15 minutes. During the spraying, the hand numbness completely disappeared and the arm pain started to disappear. After a 20-minute pause of the spraying, Brown's gas was again sprayed on the shoulder and arm for 15 minutes. The shoulder and arm pain was remarkably alleviated so that the arm could move freely. After 45 minutes, Brown's gas was again sprayed on the shoulder and the disc regions for 15 minutes. The pain completely disappeared and color was regained.

(6) At the next day after the treatment, the shoulder was remarkably relaxed but there was slight pain in the movement of the arm.

### Example 3

Subject: female with the age of 68

Symptoms: degenerative arthritis of two knees: severe pain and stiffness in standing and walking, and numbness.

Treatment and Effect:

(1) Brown's gas was sprayed on the right knee joint for 15 minutes and then on the left knee joint for 15 minutes. Immediately after the spraying, the patient could walk at a brisk pace and the pain completely disappeared. The patient could walk on a flat ground with no assistance and felt no pain when she went up and down the stairs.

(2) After a 20-minute pause of the spraying, Brown's gas was additionally sprayed on the right and left knee joints (5 minutes for each).

(3) At the next day of the treatment, there was no pain in the left knee joint but pain was felt in the right knee joint.

(4) At four weeks after the treatment, the therapeutic effects were maintained to an extent that the left knee joint was relaxed and no pain was felt.

### Example 4

Subject: female with the age of 76

Symptoms: degenerative arthritis of the left knee: remarkably swollen knee joint. In particular, severe pain due to contact between the two bones of the knee joint in walking at stairs or a slope.

Treatment and Effect:

(1) Brown's gas was sprayed on the left knee joint remarkably swollen by inflammation for 15 minutes. Immediately after the spraying, the knee joint was relaxed and the pain completely disappeared, and the patient could walk at a brisk pace. The patient could walk on a flat ground with no assistance and did not feel severe pain when she went up and down the stairs. Also, it was visually observed that the swelling subsided at the knee joint swollen by inflammation.

(2) After a 20-minute pause of the spraying, Brown's gas was again sprayed on the knee joint for 15 minutes. Additionally, the spraying of Brown's gas was repeated twice in the same manner.

(3) At the next day after the treatment, the pain of the knee joint in walking was remarkably alleviated, showing that the therapeutic effect was maintained during the post-treatment period.

### Example 5

Subject: female with the age of 50

Symptoms: severe headache above 10 years.

Treatment and Effect:

(1) Brown's gas was sprayed on the back part of the neck for 15 minutes and on the both shoulders for 10 minutes (for each). During the spraying, the back part of the neck was relaxed and disappearance of headache was felt. After the spraying, the headache disappeared. In addition, the dimness and redness in the bloodshot eyes were removed.

(2) At the next day after the treatment, slight headache was felt.

(3) At two weeks after the treatment, only slight headache was felt.

### Example 6

Subject: the same person in Example 2

Symptoms: stretched ligament of the right foot: severe pain and stiffness in walking.

Treatment and Effect:

(1) Brown's gas was sprayed on the right foot for 5 minutes. Immediately after the spraying, the patient stood up and walked, and no pain was felt.

(2) At the next day after the treatment, no pain was felt in the right foot in walking.

(3) At ten days after the treatment, the ligament of the right foot was completely cured.

### Example 7

Subject: male with the age of 45

Symptoms: right shoulder shock during exercising. In office hours, frozen shoulder spread to the neck so that the neck could not be bent back. Reddish neck due to allergic symptoms at the neck skin.

Treatment and Effect:

(1) Brown's gas was sprayed on the shoulder and neck for 15 minutes. The pain completely disappeared. Brown's gas was again sprayed on the feet for 5 minutes.

(2) Immediately after the spraying, the patient stood up and walked and felt no pain. In addition, the allergic symptoms at the neck skin completely disappeared and thus the neck skin was restored to normal color.

(3) At the next day after the treatment, no pain was felt and the neck and shoulder was restored to a normal condition.

(4) At the three days after the treatment, slight pain was felt in the affected shoulder but there was no trouble in performing daily routine activities.

(5) At 18 days after the treatment, the symptoms were completely cured.

### Example 8

Subject: male with the age of 44

Symptoms: chronic waist pain and numbness in the both big toes.

Treatment and Effect:

(1) Brown's gas was sprayed on the waist for 20 minutes. The patient felt remarkably alleviated pain.

(2) At three days after the treatment, severe waist pain was again felt.

### Example 9

Subject: male with the age of 58

Symptoms: acute stomachache.

Treatment and Effect:

(1) Brown's gas was sprayed on the abdomen with pain for 15 minutes.

(2) Unlike before the spraying in which the abdominal pain was too severe to move, immediately after the spraying, the abdominal pain completely disappeared.

### Example 10

Subject: female with the age of 47

Symptoms: severe headache above 10 years; edema in the shoulder, the neck, etc., and stiffness in walking, arm raising, and shoulder spinning due to severe pain.

Treatment and Effect:

(1) Brown's gas was sprayed on the neck for about 20 minutes. At five minutes after initiation of the spraying, the dimness of the eyes was improved. When the spraying was completed, the patient could gradually spin his neck. The head became clear and the headache disappeared.

(2) Brown's gas was sprayed on the shoulder and the chest five times (20 minutes for each). The patient experienced considerable alleviation of the pain, lightness of the body, and free motion.

### Example 11

Subject: male with the age of 51

Symptoms: good condition and no particular disease, but dim eyes when tired.

Treatment and Effect:

(1) Brown's gas was sprayed on the neck for about 15 minutes. The dimness of the eyes started to disappear. Small characters at far distance started to be viewed.

(2) At the next day after the treatment, activation of the body was felt. In spite of insufficient sleeping, no fatigue was felt and eyesight was regained.

### Example 12

Subject: female with the age of 45

Symptoms: acute waist pain upon waking. Even during sleeping, the waist pain was too severe to move.

Treatment and Effect:

(1) Brown's gas was intensively sprayed on the waist pain region for 25 minutes. At the night of the even day (at 6 hours after the spraying), the spraying of Brown's gas on the same region was additionally performed for 15 minutes.

(2) Until the next day after the treatment, the patient felt no pain during sleeping and had a sound sleep. No waist pain upon waking was felt.

### Example 13

Subject: male with the age of 48

Symptoms: acute waist pain and systemic myalgia due to unexpected physical labor (mowing); pain in head shaking and discomfort in sleeping and movement.

Treatment and Effect:

(1) Lumbago: Brown's gas was sprayed on the pain region for about 20 minutes.

(2) Myalgia: Brown's gas was sprayed on the shoulder and the muscles and ligaments that support the spine for about 10 minutes.

(3) Immediately after the spraying, the lumbago was alleviated and the myalgia was cured so that the head could be freely shaken. After the treatment, the symptoms were continuously improved with time. At six hours after the treatment, no pain was felt.

(4) At two days after the treatment, the symptoms of both the lumbago and myalgia disappeared and no pain was felt. It was considered that the patient was completely cured.

(5) As a result of the intermittent examinations during one month after the treatment, it was concluded that the patient was completely cured.

### Example 14

Subject: male with the age of 51

Symptoms: disc symptoms: numbness in the both hands (usual), continued loss of sensation (often); frozen shoulder was too severe to sleep; stiffness and pain in the neck.

Treatment and Effect:

(1) Brown's gas was sprayed on the neck, shoulder, the fifth and sixth cervical vertebrae, and the waist for about 10-15 minutes (for each).

(2) At five minutes after initiation of the spraying on the cervical vertebrae, the numbness of the both hands was gradually alleviated. Within one hour after the termination of the spraying, the numbness of the hands completely disappeared. Sensation of the hands was recovered. Further, during the spraying, the head became clear.

(3) As a result of the intermittent examinations during one month after the treatment, it was concluded that the numbness of the hands due to the disc completely disappeared.

### Example 15

Subject: male with the age of 55

Symptoms: muscular sclerosis of the shoulder and neck above 10 years: severe headache and stiffness in the neck and shoulder.

Treatment and Effect:

(1) Brown's gas was once sprayed on the shoulder and neck for 15 minutes. The neck was relaxed and the headache disappeared.

(2) At 30 minutes after the treatment, Brown's gas was again sprayed on the same regions for 15 minutes. At 30 minutes after the treatment, Brown's gas was again sprayed on the same regions for 10 minutes. The eyesight was regained and the redness of the bloodshot eyes before the spraying disappeared, like in use of an eye lotion. The shoulder and neck were remarkably relaxed so that the patient could behave naturally, and the pain almost completely disappeared. Further, the patient became energetic so that he did not feel fatigue.

(3) As a result of the intermittent examinations during two months after the treatment, it was concluded that the headache completely disappeared.

### Example 16

Subject: male with the age of 48

Symptoms: continued lumbago and neck myalgia through overwork for recent several days; prescription drugs or medicines had not yet been received.

Treatment and Effect:

(1) Brown's gas was sprayed on the lumbago region for 20 minutes and then on the neck myalgia region for 5 minutes. Immediately after the spraying, the pain completely disappeared. The patient did not feel discomfort and became energetic.

(2) At the next day after the treatment, no symptoms of the lumbago and the neck myalgia were observed. The patient became energetic and felt a good condition.

### Example 17

Subject: male with the age of 68

Symptoms: dwarfness; numbness and loss of sensation in the left leg due to the waist disc; pain of the right hand (frozen shoulder, elbow arthralgia) due to the cervical disc; and arthritis in the both knees.

Treatment and Effect:

(1) First, Brown's gas was sprayed on the cervical disc region (fifth and sixth cervical vertebrae) for 10 minutes. The pain was felt in the disc region before the spraying, whereas the pain disappeared at five minutes after the spraying. The frozen shoulder also gradually disappeared.

(2) Brown's gas was sprayed on the elbow with severe pain for 10 minutes. The pain gradually disappeared. Brown's gas was again continuously sprayed on the elbow for 10 minutes.

(3) At the next day after the treatment, Brown's gas was sprayed on the right knee joint for 15 minutes. Immediately after the spraying, the patient could go up and down the stairs with no trouble. Brown's gas was sprayed on the left knee joint for 15 minutes. The left foot became warm and the pain of the knee joint disappeared. After 20 minutes, Brown's gas was continuously sprayed on the waist (lumbar vertebrae) for 15 minutes. After the spraying, the patient could easily go up and down the stairs. The color of the patient was regained and the body became warm. The patient, who had been considered as “hopeless case” before the treatment, experienced remarkably alleviated pain and natural behavior after the treatment.

(4) At two days after the treatment, the following improvements were observed.
Disappearance of the pain in the right shoulder and arm due to the disc.
Complete disappearance of numbness in the left leg.
Disappearance of the arthritis symptoms in the both knees and no stiffness in walking at stairs.

### Example 18

Subject: male with the age of 56

Symptoms: pain due to waist disc dislodgement; stiffness in usual walking; and numbness in the left leg.

Treatment and Effect:

(1) First, Brown's gas was sprayed on the waist (lumbar vertebrae) for 20 minutes. Unlike before the spraying, the patient could easily rise from his seat after the spraying. After a 20-minute pause of the spraying, Brown's gas was again sprayed on the same region for 30 minutes. After 1.5 hours, Brown's gas was again sprayed on the same region for 30 minutes.

(2) At the next day of the treatment, the numbness of the left leg disappeared and the waist pain was also remarkably alleviated.

### Example 19

Subject: male with the age of 74

Symptoms: severe pain of the right knee joint; sciatica; severe knee pain and severe stiffness in walking.

Treatment and Effect:

(1) Brown's gas was sprayed on the knee for 15 minutes. After the spraying, the knee pain completely disappeared. The patient could rise from his seat and go up and down the stairs with no assistance. After a 20-minute pause of the spraying, Brown's gas was sprayed on the waist for 20 minutes. After the spraying, the legs were relaxed and little stiffness in walking was felt.

(2) At the next day of the treatment, the therapeutic effects were maintained and thus no pain was felt.

### Example 20

Subject: male child with the age of 10

Symptoms: myasthenia, which is presently known as an incurable disease: no muscular growth, muscle weakness, and lying down through life.

Treatment and Effect:

(1) Spraying of Brown's gas was focused on stretching of the contracted ligament of the heels inhibiting walking to achieve muscle relaxation. Primarily, Brown's gas was sprayed on the ligament of the right heel for about 20 minutes. During the spraying, to correct a bouncy walk, the ankles were allowed to be bent forward. After a 20-minute pause of the spraying, the same treatment was applied to the ligament of the left heel. The patient could stand up only for less than three seconds before the spraying, whereas he could stand up for more than 50 seconds after the spraying. The two arms, which had been usually cold before the spraying, became warm. The patient felt energy.

(2) At the next day of the treatment, it was reported that the patient had a sound sleep. However, unlike on the day of the treatment, the patient could not stand up for a long time. However, rehabilitation could be made easier.

(3) At two days after the treatment, a secondary treatment was performed in the same manner as in the primary treatment. The patient could stand up for more than 50-60 seconds after the spraying, like in the primary treatment.

(4) At four days after the secondary treatment, rehabilitation could be made much easier.

### Example 21

Subject: male with the age of 53

Symptoms: Parkinson's disease: right hand/foot paralysis. The patient was under paralysis therapeutic drug intake (4-5 times per day).

Treatment and Effect:

1. Primary Treatment

(1) Brown's gas was sprayed on the neck (cervical vertebrae) and the head for 10 minutes and then on the right shoulder for 20 minutes. During the spraying, the paralysis symptoms of the right shoulder and arm started to disappear. Also, the paralysis symptoms of the right leg almost completely disappeared (85% recovery rate). The patient could stand up and walk and spin the shoulder, like a normal person. After a 30-minute pause of the spraying, Brown's gas was again sprayed on the same regions for 20 minutes.

(2) During a 1.5-hour pause of the spraying, slight paralysis symptom appeared. After initiating an exercise, the paralysis disappeared.

2. Secondary Treatment

(1) At five hours after the primary treatment, Brown's gas was sprayed on the same regions for 30 minutes. At five minutes after initiation of the spraying, the paralysis symptom of the arm completely disappeared. At 10 minutes after initiation of the spraying, the paralysis symptom of the leg also completely disappeared.

(2) In comparison between the primary and secondary treatments, the patient condition after the secondary treatment was much improved, relative to that after the primary treatment. The paralysis completely disappeared and 100% normal condition was recovered.

(3) At four hours after the secondary treatment, the normal condition was maintained.

### Example 22

Subject: female with the age of 45

Symptoms: left frozen shoulder and left head migraine

Treatment and Effect:

(1) Brown's gas was sprayed on the left shoulder for 30 minutes. At 10 minutes after initiation of the spraying, the left shoulder was relaxed. During the continued spraying, the migraine disappeared and the left head was relaxed. At 30 minutes after initiation of the spraying, the pain completely disappeared and the head became clear.

(2) At the next day of the treatment, the head became clear, the migraine disappeared, and the left shoulder was relaxed.

(3) At the three days of the treatment, slight pain was felt but the patient condition was improved by 70% or more.

### Example 23

Subject: male with the age of 59

Symptoms: gout: dark blue and swollen right foot, paralysis of the bottom of the foot, and severe pain; diabetic peripheral neuropathy; pain in the right shoulder: difficulty in arm spinning and movement, and severe pain; and rheumatoid arthritis in the both hands.

Treatment and Effect:

(1) Brown's gas was sprayed on the shoulder and cervical vertebrae (fifth and sixth cervical vertebrae) for 25 minutes and 5 minutes, respectively. During the spraying, the frozen shoulder completely disappeared. Also, the finger pain of the both hands disappeared and the both hands were relaxed.

(2) Brown's gas was sprayed on the top side of the left foot for about 30 minutes. At 2-3 minutes after initiation of the spraying, the swelling of the swollen top side of the foot subsided remarkably and the dark blue skin color was also changed to a normal skin color. The severe pain was alleviated and the paralysis disappeared. Immediately after the spraying, the patient could rise from his seat and walk with no pain.

(3) At the next day (at 15 hours) after the treatment, it was reported that the patient had a sound sleep, the right frozen shoulder completely disappeared, and the swollen left foot was restored to a normal condition. Pain was felt at an upper part of the leg joint but was at a remarkably alleviated level relative to the pain before the treatment. These effects are attributed to the function of Brown's gas as an antioxidant reducing the level of uric acid which is a causative substance of gout.

(4) At three hours, no frozen shoulder was felt and the shoulder was significantly relaxed. The right foot was also maintained at normal condition. With respect to the right leg on which Brown's gas had not been sprayed, the pain was alleviated and the swelling disappeared. Even though Brown's gas was sprayed on only the shoulder and neck, the pain of the both hands disappeared. Even though Brown's gas was sprayed on only the left leg, the therapeutic effect was also observed on the right leg. The phenomena might be possible because Brown's gas does not stay in its application region but is absorbed in the body and then circulated through the blood stream in the body.

### Example 24

Subject: male with the age of 80

Symptoms: herpes shingles; severe pain like bee stinging, stinging and severe pain even when the hands or clothes brushed against the pain region, and hard life due to continued pain for three years.

Treatment and Effect:

(1) Primarily, Brown's gas was sprayed on the back with pain for 40 minutes. The pain was alleviated by 70%.

(2) Secondarily, Brown's gas was sprayed on the chest for 30 minutes. The pain was remarkably alleviated.

(3) Brown's gas was again sprayed on the back. The pain was considerably alleviated so that little pain was felt, as compared to the primary and secondary spraying. Next, Brown's gas was sprayed on the left chest. The right chest pain completely disappeared.

(4) The hands and feet became warm by improved blood circulation of the body. Further, unlike before the treatment, a light body condition was maintained.

### Example 25

Subject: female with the age of 81

Symptoms: herpes shingles, waist pain, and sciatica

Treatment and Effect:

(1) Brown's gas was sprayed on the waist (lumbar vertebrae) for 30 minutes. The patient suffered from waist and leg pain in walking due to sciatica before the treatment, whereas the left pelvis and knees were relaxed, no pain was felt, and no discomfort was felt in walking, after the treatment.

(2) Brown's gas was also sprayed on the herpes shingles region. The feeling of relaxation was felt and the pain was remarkably alleviated.

### Example 26

Subject: male with the age of 44

Symptoms: eye's movement relating nerve disorder; 10 years ago, inflammation formed in the back part of the head, in which sensory neurons are distributed, affected the optic nerve, thereby resulting in stiffness in facial muscle, stiffness and redness in the eyes.

Treatment and Effect:

(1) Brown's gas was sprayed on the back part of the neck for 30 minutes. At five minutes after the spraying, stiffness in the eyes was remarkably alleviated. Also, the facial muscle was relaxed and the redness in the eyes was removed.

(2) At the next day after the treatment, the patient still maintained improved conditions even though the improved conditions did not reach the improved conditions immediately after the treatment. It was expected that continued treatment would result in remarkable improvement.

(3) At two days after the treatment, the condition of the eyes was still good. Even when the condition of the eyes became bad, it was rapidly recovered by a good rest, unlike before the treatment.

### Example 27

Subject: male with the age of 39

Symptoms: tonsillitis due to a cold: difficult to swallow saliva and pain in the left shoulder and the right arm.

Treatment and Effect:

(1) Brown's gas was sprayed on the neck for 30 minutes. The symptoms were improved so that the patient could swallow saliva. The patient was restored to a normal condition by complete removal of the pain by the continued spraying.

(2) To alleviate the frozen shoulder, Brown's gas was continuously sprayed on the neck for 20 minutes. The right shoulder felt the symptoms like electric shock and the frozen shoulder disappeared.

(3) Even after 24 hours of the treatment, the improved condition of the patient was still maintained.

### Example 28

Subject: male with the age of 38

Symptoms: chronic headache and panic disorder associated with depression. The patient was under daily drug intake.

Treatment and Effect:

1. Primary Treatment

(1) Brown's gas was sprayed on the neck for 30 minutes. In the initial stage of the spraying, dizziness were transiently felt but gradually disappeared. The patient gradually had a clear head. Because the patient took medicine on the morning of the day of the treatment, to identify the therapeutic effects of the Brown's gas took much time.

(2) At the next day after the treatment, the head still maintained a clear condition and no headache was felt.

(3) At two days after the treatment, even though drug intake was discontinued except the morning of the first day of the treatment, the therapeutic effects of a clear head and no headache were still maintained and panic disorder was not observed. During the post-treatment period, disorder symptoms were slightly observed for about 10 minutes but the patient was rapidly recovered from the disorder symptoms. As can also be seen in another Examples, the increase in the recovery power of the body might be possible because Brown's gas does not transiently alleviate symptoms but returns a lesional region of interest, i.e., lesional tissue or organ, to a normal condition. Therefore, it is thought that the tissue or organ itself has a self-recovery power.

(4) At three days after the treatment, no headache or panic disorder was observed.

(5) In the morning of the fourth day after the treatment, slight headache was observed but almost completely disappeared after sound sleep for one hour.

2. Secondary Treatment

(1) In the evening of the fourth day after the primary treatment, Brown's gas was sprayed on the back part of the neck for 25 minutes. The slight pain completely disappeared. After about one hour, Brown's gas was again sprayed on the same region for 10 minutes. The condition of the patient was completely recovered.

(2) At the next day after the treatment, all the disorder symptoms disappeared to become normal like a healthy person. In the afternoon of the day, slight headache was felt. However, it was considered not to be associated with the panic disorder. It might be the aftereffect of the discontinuation of the drug intake.

(3) At three days after the secondary treatment, according to the doctor's diagnosis, the feeling of slight headache might be the aftereffect of the disconnection of the drug intake and would disappear after about 10 days.

3. Tertiary Treatment

(1) At 23 days after the secondary treatment, Brown's gas was sprayed on the back side of the head for 30 minutes. Like in the primary and secondary treatments, the head became clear and the body became energetic.

(2) At the next day after the treatment, no headache was felt and a good condition was maintained. The patient felt like a healthy person.

(3) It was judged that if the patient would be subjected to continued treatment at least once a month, he could be returned to a normal condition.

### Example 29

Subject: male with the age of 42

Symptoms: sore throat and much phlegm due to much smoking (usual).

Treatment and Effect:

(1) Brown's gas was sprayed on the neck for 10 minutes. During the spraying, the throat felt itchy and prickly.

(2) At the next day after the treatment, the soreness of the throat and phlegm disappeared and the neck was relaxed.

### Example 30

Subject: male child with the age of 8

Symptoms: rhinitis from an early age.

Treatment and Effect:

(1) Brown's gas was directly sprayed into the nose for five minutes. The nose was no longer blocked and the concentrated nasal mucus was continuously swallowed.

(2) At the next day after the treatment, Brown's gas was sprayed into the nose for five minutes. The same effect as in the previous day was observed. It was judged that if the patient might be subjected to continued treatment, remarkable improvements or therapeutic effects would be achieved.

### Example 31

Subject: male with the age of 46

Symptoms: Achilles myositis: pain due to the Achilles myositis of the left ankle above one week. Even though the patient was subjected to oriental medicinal treatment and orthopedic treatment, remarkable therapeutic effects were not obtained. The patient walked with a limp and felt severe pain.

Treatment and Effect:

(1) Brown's gas was sprayed on the Achilles' tendon for about 10 minutes. At 2-3 minutes after the spraying, severe pain started to disappear and the swelling subsided. After the spraying was completed, the pain almost completely disappeared.

(2) At three days after the treatment, the disorder symptoms were again observed. It might be thought that the therapeutic effects were not maintained due to overwork after the treatment.

(3) Brown's gas was again sprayed on the same region for 10 minutes. Immediately after the spraying, the pain disappeared. The improved therapeutic effects relative to the primary treatment were felt.

(4) At the next day after the treatment, the pain completely disappeared and the therapeutic effects were maintained.

### Example 32

Subject: female with the age of 70

Symptoms: cervical disc: frozen shoulder and hand numbness; severe pain in the top side of the left foot (the pain was too severe to put on her shoes due to an injury caused by falling down 10 years ago); and burning feeling due to gastritis (usual).

Treatment and Effect:

(1) First, to alleviate the frozen shoulder due to the cervical disc, Brown's gas was sprayed on the neck for 20 minutes. During the spraying, stiffness and the feeling of pain in the joints were felt. The hand numbness and the frozen shoulder disappeared.

(2) After a 30-minute pause of the spraying, Brown's gas was sprayed on the top side of the left foot for 20 minutes. During the spraying, pain transiently appeared in the toes but disappeared. The patient could stand up and walk with no pain and discomfort.

(3) Brown's gas was sprayed on the abdomen (the stomach region) for 15 minutes. During the spraying, slight pain was felt in the stomach. The pain gradually became worse but disappeared. Gas was incessantly generated in the stomach.

(4) At the next day after the treatment, the pain in the top side of the left foot completely disappeared so that the left foot was restored to a normal condition. The frozen shoulder was remarkably alleviated but was not completely cured. However, the patient could have a sound sleep. Judging from the observation that gas was incessantly generated in the stomach, the gastritis was not improved.

(5) At three days after the treatment, the body condition was remarkably improved and no pain in the top side of the left foot and the shoulder was felt. With respect to the gastritis, gas generation did not occur. The patient had a comfort feeling in the stomach and did not feel other disorder symptoms.

(6) At seven days after the treatment, other disorder symptoms were not felt and thus the patient felt that the body was completely restored to a normal condition. Also, the patient had the feeling of comfort in the stomach and had no trouble in digestion. In addition, it was felt that constipation was removed.

### Example 33

Subject: female with the age of 35

Symptoms: chronic gastritis (doctor's diagnosis) for seven years.

Treatment and Effect:

(1) Brown's gas was sprayed on the abdomen for about 15 minutes. During the spraying, the patient felt icy pain and gas generation in the stomach.

(2) At two days after the treatment, the patient had the feeling of comfort in the stomach. At three months after the treatment, the therapeutic effects were still maintained.

### Example 34

Subject: female with the age of 62

Symptoms: cataract and glaucoma as complications of chronic diabetes.

Treatment and Effect:

(1) Brown's gas was sprayed on the eyes for about 20 minutes. During the spraying, the eyes were gradually relaxed and small characters at far distance were vividly viewed and the therapeutic effects were maintained.

(2) During two days after the treatment, Brown's gas was additionally sprayed on the eyes once a day. Continued improvement in the therapeutic effects was observed.

### Example 35

Subject: female with the age of 15

Symptoms: epidemic keratoconjunctivitis: swollen, itchy, blear, and bloodshot eyes.

Treatment and Effect:

(1) Brown's gas was sprayed on the affected eyes for about 10 minutes. During the spraying, the ocular disease symptoms gradually disappeared and the therapeutic effects were maintained.

(2) At 10 hours after the spraying, a secondary spraying was performed. Residual redness in the bloodshot eyes disappeared and the eyes of the patient were restored to a normal condition.

### Example 36

Subject: male with the age of 45

Symptoms: stricture of the heart: stiffness and severe pain in the left chest and difficulty in breathing.

Treatment and Effect:

(1) Brown's gas was sprayed on the affected left chest for about 30 minutes. During the spraying, the pain was gradually alleviated and breathing was comfortable.

(2) The symptoms were alleviated by 70-80%.

### Example 37

Subject: female with the age of 36

Symptoms: burn on the shoulder: scabs formed during healing the burn, and severe itch.

Treatment and Effect:

(1) Brown's gas was sprayed on the affected region for about 30 minutes. The itchy symptom gradually disappeared. The therapeutic effect was maintained for more than 12 hours after the treatment.

(2) Whenever the itchy symptom was felt, Brown's gas was sprayed on the affected region. It was observed that wound recovery was accelerated.

(3) Brown's gas was also sprayed on an affected region with similar symptoms to a burn, which had been caused after laser therapy for elimination of points on the face, in the same manner as in sections (1) and (2). In this case, the itchy symptom completely disappeared and the wound was healed without leaving scars.

### Example 38

Subject: male with the age of 19

Symptoms: crack of the right wrist by falling down in a hurdle race; casting and frequent numbness in the hands.

Treatment and Effect:

(1) Whenever the numbness in the hands occurred, Brown's gas was sprayed on the tips of the fingers and the elbow for about 10 minutes. During the spraying, the numbness symptom disappeared and the wrist was relaxed. The therapeutic effects were maintained during a prolonged period of time after the spraying.

(2) At three months after the treatment, the casting was removed. However, since the fingers and wrist were not used for a long time, stiffness and pain were felt. Brown's gas was again sprayed on the knuckles and the affected wrist for three days three times. Even though the doctor in charge diagnosed that rehabilitation had to be made for about one month for recovery, the stiffened wrist was easily relaxed only by the three times spraying of Brown's gas and almost completely restored to a normal condition.

### Example 39

Subject: male with the age of 67

Symptoms: Parkinson's disease over 17 years

Treatment:

Brown's gas was 30 times (total) sprayed on the affected regions for 27 days ranging from Jun. 15, 2004 to Jul. 11, 2004. The treatment with Brown's gas was performed together with drug intake. As a result, initial and primary symptoms of the Parkinson's disease and its secondary symptoms including complications could be cured. In this regard, it was judged that the patient could have a normal life.

Effects: following post-treatment symptoms in the patient were provided based on the subjective symptoms of the patient.
Reduction in the number of drug intake and dosage: reduction from 6 times to 3-4 times.
Improvement in bradykinesia (slow motion).
Improvement in stiffness: pain in the neck, shoulder, and joints disappeared and were relaxed.
Improvement in walking disorder: the maximum therapeutic effect was that the patient was restored to a normal condition so that he could jump about.
Improvement in sense of equilibrium: no falling down in walking.
Clear and bright face, loud voice, and clear pronunciation.
Improvement in swallowing: tongue or neck muscles were activated.
Improvement in saliva swallowing: the autonomic nerve was recovered and the neck muscle was activated.
Recovery of the disorder of urination: the urinary color was normal and the disorder of urination was remarkably improved.
Constipation alleviation: the motion of the intestinal tract was remarkably improved even though there was a difference in the degree of the motion of the intestinal tract according to the type of a food.
Sexual dysfunction recovery.
Improvement in eyesight: eyesight was regained so that the reading of small characters was possible.
Removal of perspiration: much sweating in the upper part of the body was improved.
Recovery of dementia disorder: the failure of the memory was improved.
Removal of depression: good feeling.
Recovery of the disorder of foot sensation: the sensation of the right foot was recovered.
Sound sleep: sleeping for 6 to 9 hours.
Headache removal: clear and light head.
Recovery of the sense of taste: good appetite and weight gain.

Most results from the above Examples were based on short-term observations. Sufficient follow-up tests and additional treatments have not yet been conducted. However, in the short-term experiments, most patients experienced complete removal of symptoms or remarkable improvements during spraying, immediately after spraying, or within 24 hours. These results demonstrate direct action of Brown's gas on the body, as described above.

Furthermore, according to the examination results for the Examples, with respect to a disease with severe symptoms (in particular, for myalgia) with a short onset period, the severe symptoms were almost completely cured by spraying occurred only once. However, with respect to a disease with mild symptoms with a long onset period, the improvement of the symptoms after the treatment was transiently observed, but with time, some symptoms were slightly again observed and some symptoms reached the same level as the original severe symptoms, which will now be described. For the former, lesional tissues, cells, or organs are easily restored to normal conditions by treatment with Brown's gas and the restored conditions are maintained during the post-treatment period. On the other hand, for the latter, even though lesional tissues, cells, or organs exhibit recovered conditions such as alleviation or disappearance of the symptoms, the recovered conditions are transient and thus return to the conditions before the treatments.

However, some diseases with a long onset period were remarkably improved by repeated treatment with Brown's gas, and remarkable increase in the recovery power of the body was observed in some of the Examples. The increase in the recovery power of the body might be possible because Brown's gas does not transiently alleviate symptoms but returns a lesional region of interest, i.e., lesional tissue or organ, to a normal condition, whereby the tissue or organ itself has a self-recovery power. These results show that diseases with severe symptoms and a long onset period can also be completely cured by repeated treatment with Brown's gas.

The therapeutic effects of Brown's gas are also observed on non-application regions of the body, in addition to application regions. This might be possible because Brown's gas does not stay in its application region but is absorbed in the body and then circulated through the blood stream in the body.

### INDUSTRIAL APPLICABILITY

Through the above-described various Examples, it can be seen that application of Brown's gas to the body induces various therapeutic effects such as muscle relaxation and pain removal in myalgia, alleviation of symptoms and pain in arthritis, symptom alleviation in intervertebral disc, inflammation prevention and cure in wounded regions, prevention of inflammatory and allergic cutaneous reaction, recovery of headache and numbness, etc. These various effects of Brown's gas on the body, which have been demonstrated by illustrative examples, are essentially associated with the action and efficacy of water in the body. According to the present invention, various diseases of mammals can be treated or alleviated using Brown's gas. Furthermore, a Brown's gas supply apparatus according to the present invention can more easily supply Brown's gas for a therapeutic purpose.

What is claimed is:

1. A method of treating or alleviating the symptoms of a lesion in a mammal, comprising spraying locally a Brown's gas on a lesional area comprising tissue, cell, organ of the mammal, its related region, and its adjacent skin.

2. The method of claim 1, wherein the lesion is one selected from the group consisting of myalgia, arthritis, rheumatoid arthritis, disc, infectious inflammation, non-infectious inflammation, allergy, rhinitis, bronchitis, asthma, myasthenia, neuralgia, headache, ocular disease, Parkinson's disease, gout, panic disorder, diabetic peripheral neuropathy, blood circulation dysfunction, cardiovascular disease comprising stricture of the heart, pain, moisture deficiency, and dehydration.
